(19) United States
(12) Patent Application Publication
(10) Pub. No.: US 2021/0228496 A1
(43) Pub. Date: Jul. 29, 2021

(54) METHOD FOR REDUCING SKIN SENSITIZATION OF AN ASENAPINE-CONTAINING PATCH

(71) Applicant: HISAMITSU PHARMACEUTICAL CO., INC., Tosu-shi, (JP)

(72) Inventors: Yuka TAKAGI, Tsukuba-shi (JP); Kazuya ISHIDA, Tsukuba-shi (JP); Takao KUROKAWA, Tsukuba-shi (JP); Yasunari MICHINAKA, Tsukuba-shi (JP)

(73) Assignee: HISAMITSU PHARMACEUTICAL CO., INC., Tosu-shi (JP)

(21) Appl. No.: 16/751,307

(22) Filed: Jan. 24, 2020

(51) Int. Cl. A61K 9/70; A61K 47/06; A61K 31/407

(52) U.S. Cl. CPC A61K 9/7053; A61K 31/407; A61K 47/06

(57) ABSTRACT

A method for reducing skin sensitization of an asenapine-containing patch comprising a backing layer and an adhesive agent layer, the method comprising:

using an adhesive agent for preparing the adhesive agent layer, the adhesive agent comprising free asenapine in a range of 2 to 5% by mass, isopropyl palmitate in a range of 5 to 12% by mass, styrene-isoprene-styrene block copolymer in a range of 10 to 20% by mass, polyisobutylene in a range of 3 to 10% by mass, alicyclic saturated hydrocarbon resin in a range of 50 to 70% by mass, and liquid paraffin in a range of 5 to 12% by mass, relative to a total amount of the adhesive agent; and

preparing the asenapine-containing patch having an amount of the adhesive agent layer after drying in a range of 100 to 400 g/m².

### BACKGROUND OF THE INVENTION

### Field of the Invention

The present invention relates to a method for reducing skin sensitization of an asenapine-containing patch comprising a backing layer and an adhesive agent layer.

### Related Background Art

Asenapine (trans-5-chloro-2-methyl-2,3,3a,12b-tetrahydro-1H-dibenzo[2,3:6,7]oxepino[4,5-c]pyrrole) is a compound having a central nervous system (CNS) suppressing activity, an anti-histamine activity, and an anti-serotonin activity and is known as a drug used in the treatment of central nervous system diseases such as schizophrenia.

As asenapine-containing pharmaceutical formulations, for example, WO2010/127674A1 describes topical formulations such as sprays, aerosols, patches, and ointments. In addition, US2015/0202183A1, US2015/0231250A1, US2015/0164862A1, US2017/0035732A1, US2017/0172981A1, US2018/0289631A1, US2018/0339050A1, US2018/0360968A1, US2019/0105391A1, and US2019/0343800A1 describe patches containing asenapine.

### SUMMARY OF THE INVENTION

The present inventors found a problem that even when the amount of the drug transferred at the application of a patch containing asenapine as a drug is substantially at the same level, there are cases where sensitization caused by the drug against the skin is observed and cases where no such sensitization is observed.

The present invention has been made in view of the above-described problem and an object of the present invention is to provide a method, in a patch containing asenapine as a drug, for reducing sensitization caused by the drug against the skin while maintaining the amount of the drug transferred at the application of the patch at a high level.

The present inventors found that in an asenapine-containing patch comprising: an adhesive agent layer that is obtained using an adhesive agent having a specific composition; and a backing layer, the amount of the adhesive agent layer laminated on the backing layer has a relation with sensitization caused by the drug against the skin while not largely influencing the amount of the drug transferred at the time of application of the patch. The present inventors then found that it was possible to achieve the above-described object by setting the amount of the adhesive agent layer within a predetermined range in the asenapine-containing patch and finally completed the present invention.

The present invention is a method for reducing skin sensitization of an asenapine-containing patch comprising a backing layer and an adhesive agent layer, the method comprising:

using an adhesive agent for preparing the adhesive agent layer, the adhesive agent comprising free asenapine in a range of 2 to 5% by mass, isopropyl palmitate in a range of 5 to 12% by mass, styrene-isoprene-styrene block copolymer in a range of 10 to 20% by mass, polyisobutylene in a range of 3 to 10% by mass, alicyclic saturated hydrocarbon resin in a range of 50 to 70% by mass, and liquid paraffin in a range of 5 to 12% by mass, relative to a total amount of the adhesive agent; and

preparing the asenapine-containing patch having an amount of the adhesive agent layer after drying in a range of 100 to 400 g/m².

According to the present invention, it is possible in an asenapine-containing patch to reduce sensitization caused by the drug against the skin while maintaining the amount of the drug transferred at the time of application of the patch at a high level.

### DETAILED DESCRIPTION OF THE PREFERRED EMBODIMENTS

Hereinafter, the present invention is described in further detail according to the preferred embodiments.

The present invention is a method for reducing skin sensitization of an asenapine-containing patch comprising a backing layer and an adhesive agent layer, the method comprising:

using an adhesive agent for preparing the adhesive agent layer, the adhesive agent comprising free asenapine in a range of 2 to 5% by mass, isopropyl palmitate in a range of 5 to 12% by mass, styrene-isoprene-styrene block copolymer in a range of 10 to 20% by mass, polyisobutylene in a range of 3 to 10% by mass, alicyclic saturated hydrocarbon resin in a range of 50 to 70% by mass, and liquid paraffin in a range of 5 to 12% by mass relative to a total amount of the adhesive agent; and

preparing the asenapine-containing patch having an amount of the adhesive agent layer after drying in a range of 100 to 400 g/m².

The asenapine-containing patch according to the present invention comprises a backing layer and an adhesive agent layer disposed on at least one surface of the backing layer. As the backing layer, a conventionally-known one may be used as appropriate, and the material of such a backing layer includes, for example, synthetic resins such as polyethylene, polypropylene, polybutadiene, ethylene-vinyl acetate copolymer, vinyl acetate-vinyl chloride copolymer, polyvinyl chloride, polyamides (such as nylon), polyester, cellulose derivatives, and polyurethane. In addition, the form of the backing layer includes a film; a sheet; a sheet-shaped porous body; a sheet-shaped foam; fabrics such as a woven fabric, a braided fabric, and a nonwoven fabric; stacked bodies of these; and the like. In the present invention, the thickness of the backing layer is not particularly limited, but it is usually preferable that the thickness be approximately in a range of 2 to 3000 μm.

Moreover, a release liner may be further included on a surface of the adhesive agent layer on the opposite side to the backing layer. Such a release liner may be any release liner as long as the release liner covers the adhesive agent layer until the use of the patch and can be peeled and removed off when the patch is used. Specifically, the release liner includes polyesters such as polyethylene terephthalate and polyethylene naphthalate; polyolefins such as polyethylene and polypropylene; films of polyvinyl chloride, polyvinylidene chloride, and the like; laminate films of wood-free papers and polyolefins; films of nylon, aluminum, and the like; and the like. As these release liners, it is preferable to use those which are subjected to surface coating with release agents of silicone, polytetrafluoroethylene, and the like (releasing treatment) from the viewpoint of facilitating releasing from the adhesive agent layer.

The adhesive agent for preparing the adhesive agent layer of the asenapine-containing patch according to the present invention comprises free asenapine in a range of 2 to 5% by mass, isopropyl palmitate in a range of 5 to 12% by mass, styrene-isoprene-styrene block copolymer in a range of 10 to 20% by mass, polyisobutylene in a range of 3 to 10% by mass, alicyclic saturated hydrocarbon resin in a range of 50 to 70% by mass, and liquid paraffin in a range of 5 to 12% by mass, relative to a total amount of the adhesive agent.

<Asenapine>

The asenapine according to the present invention refers to trans-5-chloro-2-methyl-2,3,3a,12b-tetrahydro-1H-dibenzo[2,3:6,7]oxepino[4,5-c]pyrrole. Asenapine has a central nervous system (CNS) suppressing activity, an anti-histamine activity, and an anti-serotonin activity and is generally known as a drug used in the treatment of central nervous system diseases such as schizophrenia.

In the present invention, such asenapine is required to be free asenapine which is in the free form. When free asenapine is used as a drug, combination with styrene-isoprene-styrene block copolymer and polyisobutylene, which are rubber-based adhesive agents described later improves the skin permeability of the drug.

The free asenapine according to the present invention may be free asenapine added at the time of production of a patch or may be generated from a pharmaceutically acceptable salt of asenapine in the adhesive agent layer from the viewpoint of the handleability and stability of the raw material. Alternatively, the free asenapine may be a mixture of these. The method for generating the free asenapine from a pharmaceutically acceptable salt of asenapine (hereinafter sometimes referred to as an asenapine salt) includes, for example, a method including blending the asenapine salt and a metal ion-containing desalting agent (neutralizing agent) in the composition of the adhesive agent layer at the time of production of a patch to desalinate the asenapine salt.

As the asenapine salt, an acid adduct is preferable from the viewpoint that the acid adduct is easily desalinated by the metal ion-containing desalting agent.

The acid includes monobasic acids such as hydrochloric acid, hydrobromic acid, and methanesulfonic acid; and polybasic acids such as fumaric acid, maleic acid, citric acid, and tartaric acid. One of these may be used alone or two or more of these may be used in combination.

In addition, the metal ion-containing desalting agent includes metal hydroxides, acetic acid metal salts, and the like. The metal includes sodium, potassium, magnesium, and the like. One of these may be used alone or two or more of these may be used in combination. Among these, the metal ion-containing desalting agent is preferably an alkali metal ion-containing desalting agent and particularly preferably sodium hydroxide and sodium acetate, from the viewpoint that they are easy to handle at the production and further improve the stability over time of the free asenapine.

In a case where the free asenapine according to the present invention is generated from the asenapine salt, the amount of the metal ion-containing desalting agent blended is preferably in a range of 0.5 to 6 equivalents and more preferably in a range of 0.75 to 4 equivalents relative to the acid-base equivalent of the asenapine salt. If the amount of the metal ion-containing desalting agent blended is less than the lower limit, it becomes difficult to generate the free asenapine in a sufficient amount, making it difficult to achieve a sufficient skin permeability. On the other hand, if the amount is more than the upper limit, the adhesive force of the adhesive agent layer tends to decrease.

In the adhesive agent according to the present invention, the content of the free asenapine needs to be in a range of 2 to 5% by mass relative to the total amount of the adhesive agent. If the content of the free asenapine is less than the lower limit, the skin permeation amount becomes insufficient, so that the area of the patch needs to be increased. On the other hand, if the content is more than the upper limit, local side effects such as skin irritation tend to occur or skin sensitization becomes likely to be observed. Moreover, from the same viewpoint, the content of the free asenapine is particularly preferably in a range of 3 to 4% by mass relative to the total amount of the adhesive agent.

<Rubber-Based Adhesive Agent>

In the adhesive agent according to the present invention, as the rubber-based adhesive agent, which is an adhesive base agent, it is necessary to use styrene-isoprene-styrene block copolymer (SIS) and polyisobutylene (PIB) in combination. Using the SIS and the PIB in combination with the free asenapine in this way makes it possible to achieve both an improvement in the releasability of asenapine from the adhesive agent layer and an improvement in the adhesive force of the adhesive agent layer.

In the adhesive agent according to the present invention, the content of the SIS needs to be in a range of 10 to 20% by mass relative to the total amount of the adhesive agent. If the content of the SIS is less than the lower limit, the adherability of the patch to the skin decreases. On the other hand, the content is more than the upper limit, the releasability of asenapine from the adhesive agent layer decreases, making it difficult to achieve a sufficient skin permeability. Moreover, from the same viewpoint, the content of the SIS is particularly preferably in a range of 12 to 17% by mass relative to the total amount of the adhesive agent.

In the adhesive agent according to the present invention, the content of the PIB needs to be in a range of 3 to 10% by mass relative to the total amount of the adhesive agent. If the content of the PIB is less than the lower limit, the adherability of the patch to the skin decreases. On the other hand, the content is more than the upper limit, the releasability of asenapine from the adhesive agent layer decreases, making it difficult to achieve a sufficient skin permeability. Moreover, from the same viewpoint, the content of the PIB is particularly preferably in a range of 4 to 8% by mass relative to the total amount of the adhesive agent.

In addition, a mass ratio between the SIS and the PIB (SIS:PIB) is preferably in a range of 4:1 to 3:2 and more preferably in a range of 3:1 to 2:1 from the viewpoint of achieving both an improvement in the releasability of asenapine from the adhesive agent layer and an improvement in the adhesive force of the adhesive agent layer. Moreover, a mass ratio between the free asenapine and the rubber-based adhesive agent (the total amount of the SIS and the PIB) (free asenapine:rubber-based adhesive agent) is preferably in a range of 1:3 to 1:15 and more preferably in a range of 1:4 to 1:8 from the viewpoint of achieving both an improvement in the releasability of asenapine from the adhesive agent layer and an improvement in the adhesive force of the adhesive agent layer.

<Absorption Enhancer>

In the adhesive agent according to the present invention, it is necessary to use isopropyl palmitate as an absorption enhancer. Using the isopropyl palmitate in combination with the free asenapine, the SIS, and the PIB in this manner makes it possible to improve the skin permeability of asenapine.

In the adhesive agent according to the present invention, the content of the isopropyl palmitate needs to be in a range of 5 to 12% by mass relative to the total amount of the adhesive agent. If the content of the isopropyl palmitate is less than the lower limit, the releasability of asenapine from the adhesive agent layer decreases, making it difficult to achieve a sufficient skin permeability. On the other hand, if the content is more than the upper limit, the absorption enhancer is likely to be separated from the adhesive agent layer to impair the adhesiveness of the adhesive agent layer and local side effects such as skin irritation are likely to occur. In addition, from the same viewpoint, the content of the isopropyl palmitate is particularly preferably in a range of 8 to 12% by mass relative to the total amount of the adhesive agent.

In addition, the mass ratio between the free asenapine and the isopropyl palmitate (free asenapine:isopropyl palmitate) is preferably in a range of 1:1 to 1:5 and more preferably in a range of 1:2 to 1:4 from the viewpoint of achieving both an improvement in the releasability of asenapine from the adhesive agent layer and a reduction of skin sensitization.

<Tackifier>

In the adhesive agent according to the present invention, it is necessary to use alicyclic saturated hydrocarbon resin as a tackifier. Using the alicyclic saturated hydrocarbon resin in combination with the free asenapine, the isopropyl palmitate, the SIS, and the PIB in this manner makes it possible to improve the adherability of the patch to the skin.

In the adhesive agent according to the present invention, the content of the alicyclic saturated hydrocarbon resin needs to be in a range of 50 to 70% by mass relative to the total amount of the adhesive agent. If the content of the alicyclic saturated hydrocarbon resin is less than the lower limit, the adherability of the patch to the skin decreases. On the other hand, if the content is more than the upper limit, the cohesive force of the adhesive agent layer decreases, making it likely that the pain during the peeling-off increases. In addition, from the same viewpoint, the content of the alicyclic saturated hydrocarbon resin is particularly preferably in a range of 50 to 65% by mass relative to the total amount of the adhesive agent.

In addition, the mass ratio between the rubber-based adhesive agent (the total amount of the SIS and the PIB) and the alicyclic saturated hydrocarbon resin (rubber-based adhesive agent:alicyclic saturated hydrocarbon resin) is preferably in a range of 1:1.7 to 1:5 and more preferably in a range of 1:2 to 1:4 from the viewpoint of achieving both suppression of decrease in the attachment force to the skin and suppression of decrease in the cohesive force of the adhesive agent layer. Moreover, the mass ratio between the free asenapine and the alicyclic saturated hydrocarbon resin (free asenapine:alicyclic saturated hydrocarbon resin) is preferably in a range of 1:10 to 1:30 and more preferably in a range of 1:15 to 1:25 from the viewpoint of suppressing decrease in the attachment force to the skin.

<Softener>

In the adhesive agent according to the present invention, it is necessary to use liquid paraffin as a softener. Using the liquid paraffin in combination with the free asenapine, the isopropyl palmitate, the SIS, the PIB, and the alicyclic saturated hydrocarbon resin in this manner makes it possible to improve the adherability of the patch to the skin and the pharmaceutical physical properties thereof.

In the adhesive agent according to the present invention, the content of the liquid paraffin needs to be in a range of 5 to 12% by mass relative to the total amount of the adhesive agent. If the content of the liquid paraffin is less than the lower limit, the adherability of the patch to the skin decreases. On the other hand, if the content is more than the upper limit, the cohesive force of the adhesive agent layer decreases, so that the adhesive agent layer or stickiness tends to remain on the skin after the peeling-off. In addition, from the same viewpoint, the content of the liquid paraffin particularly preferably in a range of 6 to 10% by mass relative to the total amount of the adhesive agent.

In addition, the mass ratio between the rubber-based adhesive agent (the total amount of the SIS and the PIB) and the liquid paraffin (rubber-based adhesive agent:liquid paraffin) is preferably in a range of 6:1 to 1:1 and more preferably in a range of 4:1 to 2:1 from the viewpoint of achieving both suppression of decrease in the attachment force to the skin and suppression of decrease in the cohesive force of the adhesive agent layer. Moreover, the mass ratio between the free asenapine and the liquid paraffin (free asenapine:liquid paraffin) is preferably in a range of 1:1 to 1:5 and more preferably in a range of 1:2 to 1:4 from the viewpoint of suppressing decrease in the attachment force to the skin.

<Other Additive and the Like>

As described above, the adhesive agent for preparing the adhesive agent layer of the asenapine-containing patch according to the present invention comprises the free asenapine, the isopropyl palmitate, the styrene-isoprene-styrene block copolymer, the polyisobutylene, the alicyclic saturated hydrocarbon resin, and the liquid paraffin in the respective predetermined ranges, but may further comprise an additive such as a stabilizer as necessary within a range that does not hinder the effect of the present invention.

Such a stabilizer includes tocopherol and ester derivatives thereof, ascorbic acid and ester derivatives thereof, dibutylhydroxytoluene, butylated hydroxyanisole, and the like. One of these may be used alone or two or more of these may be used in combination. Among these, it is more preferable to use dibutylhydroxytoluene as a stabilizer from the viewpoint of pharmaceutical physical properties and external appearance, and drug stabilizing effect. When the adhesive agent according to the present invention comprises such a stabilizer, the content of the stabilizer is preferably in a range of 0.1 to 3% by mass relative to the total amount of the adhesive agent. If the content of the stabilizer is less than the lower limit, the stability of each component in the patch tends to decrease. On the other hand, if the content is more than the upper limit, the cohesive force of the adhesive agent layer tends to decrease.

In addition, the adhesive agent for preparing the adhesive agent layer of the asenapine-containing patch according to the present invention preferably contains substantially no water. Since the adhesive agent according to the present invention mainly composed of hydrophobic components, if the content of water is more than 10% by mass, the water content tends to be separated from the adhesive agent layer to impair the adhesiveness of the adhesive agent layer. Here, containing substantially no water means that no water is intentionally added at the time of production, and the content of water measured by the Karl Fischer Method according to the Japanese Pharmacopoeia is less than 10% by mass relative to the total amount of the adhesive agent layer.

<Method for Reducing Skin Sensitization of Asenapine-Containing Patch>

In the present invention, preparing an adhesive agent layer made of the adhesive agent and further preparing the asenapine-containing patch such that the amount of the adhesive agent layer after drying is in a range of 100 to 400 g/m² makes it possible to reduce the skin sensitization caused by the drug when the obtained asenapine-containing patch is applied to the skin.

The method for preparing an adhesive agent layer made of the adhesive agent and further preparing the asenapine-containing patch such that the amount of the adhesive agent layer after drying is in a range of 100 to 400 g/m² is not particularly limited, and they may be produced by employing a conventionally-known method for producing a patch as appropriate. For example, first, the free asenapine, the rubber-based adhesive agent (the SIS and the PIB), the isopropyl palmitate, the alicyclic saturated hydrocarbon resin, and the liquid paraffin are kneaded together with a solvent in accordance with a conventional method to obtain a uniform adhesive agent composition. Then, this adhesive agent layer composition is applied onto a surface (usually, onto one surface) of the backing layer to a predetermined thickness, followed by as necessary heating to dry and remove the solvent, and the resultant is cut into a desired size, so that the asenapine-containing patch can be obtained.

In the present invention, when the adhesive agent layer composition is applied onto a surface of the backing layer, it is necessary to make the amount of the adhesive agent layer after drying in a range of 100 to 400 g/m². In the asenapine-containing patch comprising: the adhesive agent layer obtained by using the adhesive agent having the specific composition; and the backing layer as described above, making the amount of the adhesive agent layer laminated on the backing layer in the above-described range makes it possible to reduce the sensitization caused by the drug against the skin while maintaining the amount of the drug transferred at the time of application at a high level. In other words, if the amount of the adhesive agent layer is less than the lower limit, the amount of the drug transferred at the time of application decreases. On the other hand, if the amount is more than the upper limit, reduction of the sensitization caused by the drug against the skin becomes unlikely to be sufficiently achieved. In addition, from the same viewpoint, the amount of the adhesive agent layer after drying is particularly preferably in a range of 100 to 300 g/m².

Note that the solvent used to obtain the adhesive agent composition includes, for example, toluene, ethanol, methanol, ethyl acetate, and the like. One of these maybe used alone or two or more of these may be used in combination. In addition, the conditions for the heating may be selected as appropriate depending on the solvent; however, the temperature condition is preferably 60 to 120° C. in general and the heating time is preferably 2 to 30 minutes in general.

In addition, when the asenapine-containing patch is prepared, the method may further comprise a step of laminating the release liner on a surface of the adhesive agent layer on the opposite side to the backing layer. Moreover, a step of first applying the adhesive agent layer composition onto one surface of the release liner to the predetermined thickness to form the adhesive agent layer and thereafter laminating the backing layer on the surface of the adhesive agent layer on the opposite side to the release liner may be employed.

<Skin Sensitization and Method for Evaluating Skin Sensitization>

Skin sensitization is one of delayed-type hypersensitivities and is a phenomenon that causes rash on the skin through an excessive immune reaction due to a chemical substance. Even with a slight contact that does not exhibit primary irritation, the sensitized T-cells in the lymph nodes proliferate due to a matter having the sensitization potential, and when the skin touches the same chemical substance again, the recognized T-cells release lymphokine to cause skin inflammation, so that the skin sensitization becomes positive.

As methods for testing such skin sensitization, the Maximisation Test (Guinea Pig Maximisation Test (GPMT)), the Buehler Test, and the Local Lymph Node Assay (LLNA) are known, and any of these methods can be used for evaluating skin sensitization in the present invention. However, the Buehler Test is preferably employed from the viewpoint that this method allows evaluation only with the application to the skin. Note that the Maximisation Test and the Buehler Test are listed as TG406 of the “Organisation for Economic Co-operation and Development (OECD) GUIDELINE FOR TESTING OF CHEMICALS ” and the Local Lymph Node Assay is listed as TG429 of the same “GUIDELINE”.

### EXAMPLES

Although the present invention is described below in more detail based on Examples and Comparative Example, the present invention is not limited to Examples described below. Note that for an asenapine-containing patch obtained in each of Examples and Comparative Example, skin sensitization was evaluated in accordance with the Buehler Test listed as TG406 of “Organisation for Economic Co-operation and Development (OECD) GUIDELINE FOR TESTING OF CHEMICALS” and the amount of the drug transferred was also measured.

(Method for Evaluating Skin Sensitization)

For the asenapine-containing patch (test patch, size: 2 cm×2 cm) obtained in each of Examples and Comparative Example, skin sensitization was evaluated as described below in accordance with the Buehler Test using guinea pigs (sensitization group: 6 guinea pigs, control group (non-sensitization group): 6 guinea pigs).

<Sensitization>

The sensitization to the guinea pigs of the sensitization group was conducted as follows. Specifically, the dorsal scapular regions of guinea pigs were cleared of hair at Day 0 of the sensitization, and the test patches were applied onto the skins of these regions and held by an occlusive dressing for 24 hours. On the other hand, as the control group (non-sensitization group), guinea pigs that were not subjected to the above-described treatment were used as the untreated control group. Subsequently, the same treatment as that applied at day 0 of the sensitization was applied to the same dorsal scapular regions of the guinea pigs of the sensitization group at day 7 of the sensitization. Moreover, the same treatment as that applied at day 0 of the sensitization was applied to the same dorsal scapular regions of the guinea pigs of the sensitization group at day 14 of the sensitization as well.

<Challenge>

The Challenge was performed by clearing the hair of flanks of the guinea pigs of the sensitization group and the control group (non-sensitization group) at day 28 of the sensitization and applying the test patches onto the skins of the regions and held by an occlusive dressing for 24 hours.

<Evaluation>

The challenge areas were cleared of hair 24 hours after removing the test patches and the skin reaction was observed. The results of the observation were judged based on the skin reaction evaluation criterion of Draize shown in Table 1 to obtain the skin reaction scores (average values). The results thus obtained are shown in Table 2.

TABLE 1
Draize Skin Reaction Evaluation Criteria

| | | Evaluation |
|---|---|---|
| Observed Item | Degree | Value (*1) |
| Erythema and eschar | No erythema | 0 |
| formation | Very slight erythema | 1 |
| | (barely perceptible) | |
| | Well-defined erythema | 2 |
| | Moderate-to-severe erythema | 3 |
| | Severe erythema (beet | 4 |
| | redness) to slight eschar | |
| | formation (injuries in depth) | |
| Edema formation | No edema | 0 |
| | Very slight edema (barely | 1 |
| | perceptible) | |
| | Slight edema (edges of area | 2 |
| | well defined by definite raising) | |
| | Moderate edema (raised about | 3 |
| | 1 mm) | |
| | Severe edema (raised more than | 4 |
| | 1 mm and extending beyond | |
| | area of exposure) | |

(*1) Skin reaction score = The total point of the evaluation point of “erythema and eschar formation” and the evaluation point of “edema formation”

<Rechallenge>

To clarify the results obtained in the first challenge, a rechallenge test was conducted. Specifically, 1 week after the first challenge, the untreated flanks of the guinea pigs of the sensitization group used in the first challenge were cleared of hair, and the test patches were applied onto the skins of the regions and held by an occlusive dressing for 24 hours.

<Evaluation>

The challenge areas were cleared of hair 24 hours after and 48 hours after removing the test patches and the skin reaction was observed. The results of the observation were judged based on the above-described evaluation criterion to obtain the skin reaction scores (average values). The results thus obtained are shown in Table 2.

(Measurement of Amount of the Drug Transferred)

The amounts of the drug transferred for each guinea pig of the sensitization group at the time of the sensitization treatment, at the time of the first challenge treatment, and at the time of the rechallenge treatment were measured as described below.

Each patch (unused patch) blanked out into a size of 2 cm×2 cm (4 cm²) and each patch (peeled-off patch) peeled off from the guinea pig were each put into an extraction container, and 10 mL of tetrahydrofuran (THF) was added, followed by shaking for 60 minutes to extract the drug into the THF. Then, a diluent (methano1:0.01 mol/L SDS (1000-times diluted phosphoric acid) =3:1) was added to adjust the solution in the container precisely to 50 mL. Then, the solution thus obtained was filtrated using a membrane filter to obtain a test liquid. Each test liquid thus obtained was analyzed under the following conditions using a high-performance liquid chromatography (HPLC) to quantify the content of the drug in each patch. Then, the amount of the drug transferred into the guinea pig was obtained based on the following formula (1).

The amount of the drug transferred [μg/cm²]={(the content of the drug in the unused patch [μg])−(the content of the drug in the peeled-off patch [μg])}/4[cm²] (1)

The results thus obtained are shown in Table 2. Note that as the amount of the drug transferred at the time of the sensitization treatment, the average value [pg/cm²] of the amounts of the drug transferred at day 0, day 7, and day 14 of the sensitization was obtained.

<HPLC Conditions>

### Column: TSKgelODS-80TsQA5 μm (4.6 mm I.D.×150 mm)

Mobile phase: mixture liquid (methanol:0.01 mol/L SDS (1000-times diluted phosphoric acid)=3:1)
Measurement wavelength: 230 nm
Column temperature: 40° C.
Flow rate: 1.0 mL/min
Injection volume: 10 μL

### Example 1

First, 14.1 parts by mass of styrene-isoprene-styrene block copolymer (SIS), 6.1 parts by mass of polyisobutylene (PIB), 58.5 parts by mass of alicyclic saturated hydrocarbon resin (trade name: Arkon, manufactured by Arakawa Chemical Industries, Ltd.), and 8.1 parts by mass of liquid paraffin were mixed to obtain a uniform adhesive base agent composition. Next, 3.2 parts by mass of free asenapine, 10.0 parts by mass of isopropyl palmitate (IPP), 86.8 parts by mass of an adhesive base agent composition, and an appropriate amount of toluene were mixed to obtain a uniform adhesive agent layer composition. The composition of the adhesive agent layer composition (exclusive of the toluene) is described below.

| | Composition (% by mass) |
|---|---|
| Free asenapine | 3.2 |
| IPP | 10.0 |
| SIS | 14.1 |
| PIB | 6.1 |
| Alicyclic saturated hydrocarbon resin | 58.5 |
| Liquid paraffin | 8.1 |
| Total | 100.0 |

Next, this adhesive agent layer composition was applied onto one surface of a polyester film (release liner) subjected to a peeling process and having a thickness of 75 pm such that the amount of the adhesive agent layer composition after drying became 100 g/m², followed by drying at 80° C. for 15 minutes to remove toluene and form an adhesive agent layer. Next, a PET film backing layer) having a thickness of 25 μm was laminated on a surface of the adhesive agent layer on the opposite side to the release liner, followed by cutting to obtain an asenapine-containing patch.

### Examples 2 to 4 and Comparative Example 1

Asenapine-containing patches were obtained in the same manner as in Example 1 except that the adhesive agent layer composition was applied onto one surface of the polyester film (release liner) such that the amount of the adhesive agent layer composition after drying became 200 g/m² (Example 2) , 300 g/m² (Example 3) , 400 g/m² (Example 4), and 500 g/m² (Comparative Example 1), respectively.

For the asenapine-containing patch obtained in each of Examples and Comparative Example, the skin sensitization and the amount of the drug transferred were evaluated and the results thus obtained are shown in Table 2.

TABLE 2

| | | | | | | Comparative |
|---|---|---|---|---|---|---|
| | | Example 1 | Example 2 | Example 3 | Example 4 | Example 1 |
| The mass of the adhesive agent layer [g/m²] | | 100 | 200 | 300 | 400 | 500 |
| Skin sentisization test | The skin reaction score (sentisization | 0.0/0.3 | 0.0/— | 0.0/— | 0.0/— | 0.2/0.0 |
| (first challenge) | group/non-sensitization group, 24 hours after | | | | | |
| | removing the patch) | | | | | |
| | The amount of the drug transferred at the time | 185.2 | 286.9 | 285.2 | 294.4 | 281.6 |
| | of the sensitization treatment [μg/cm²] | | | | | |
| | The amount of the drug transferred at the time | 114.8 | 152.4 | 162.3 | 167.1 | 177.6 |
| | of the first challenge treatment [μg/cm²] | | | | | |
| Skin sentisization test | The skin reaction score (sentisization group, 24 | 0.0 | 0.0 | 0.0 | 0.0 | 0.2 |
| (rechallenge) | hours after removing the patch) | | | | | |
| | The skin reaction score (sentisization group, 48 | 0.0 | 0.0 | 0.0 | 0.2 | 0.3 |
| | hours after removing the patch) | | | | | |
| | The amount of the drug transferred at the time | 125.0 | 165.2 | 186.0 | 170.6 | 182.5 |
| | of the rechallenge treatment [μg/cm²] | | | | | |
| | Skin sensitization | negative | negative | negative | negative | positive |

As is clear from the results shown in Table 2, in the asenapine-containing patch comprising: the adhesive agent layer that is obtained using the adhesive agent having the specific composition in the range of the present invention; and the backing layer, in the cases where the amount of the adhesive agent layer laminated on the backing layer was set in the range of the present invention (Examples 1 to 4) , sensitization caused by the drug against the skin was obviously reduced as compared with the case where the amount of the adhesive agent layer was out of the range of the present invention (Comparative Example 1) , so that while sensitization was observed in the latter (Comparative Example 1), no sensitization was observed in the former (Examples 1 to 4).

In addition, since in all of the asenapine-containing patches obtained in Examples 1 to 4 and the asenapine-containing patch obtained in Comparative Example 1, the amount of the drug transferred was maintained at a high level, it was confirmed that the above-described reduction of skin sensitization was not attributable to reaction depending on a difference in amount of the drug transferred.

According to the present invention, it is possible in an asenapine-containing patch to reduce sensitization caused by the drug against the skin while maintaining the amount of the drug transferred at the time of application of the patch at a high level.

What is claimed is:

1. A method for reducing skin sensitization of an asenapine-containing patch comprising a backing layer and an adhesive agent layer, the method comprising:
using an adhesive agent for preparing the adhesive agent layer, the adhesive agent comprising free asenapine in a range of 2 to 5% by mass, isopropyl palmitate in a range of 5 to 12% by mass, styrene-isoprene-styrene block copolymer (SIS) in a range of 10 to 20% by mass, polyisobutylene (PIB) in a range of 3 to 10% by mass, alicyclic saturated hydrocarbon resin in a range of 50 to 70% by mass, and liquid paraffin in a range of 5 to 12% by mass, relative to a total amount of the adhesive agent; and
preparing the asenapine-containing patch having an amount of the adhesive agent layer after drying in a range of 100 to 400 g/m².

2. The method for reducing skin sensitization according to claim 1, wherein
the amount of the adhesive agent layer after drying is in a range of 100 to 300 g/m².

3. The method for reducing skin sensitization according to claim 1, wherein
a mass ratio between the SIS and the PIB (SIS:PIB) is in a range of 4:1 to 3:2.

4. The method for reducing skin sensitization according to claim 1, wherein
a mass ratio between the free asenapine and a total amount of the SIS and the PIB (free asenapine:(SIS+PIB)) is in a range of 1:3 to 1:15.

5. The method for reducing skin sensitization according to claim 1, wherein
a mass ratio between the free asenapine and the isopropyl palmitate (free asenapine:isopropyl palmitate) is in a range of 1:1 to 1:5.

6. The method for reducing skin sensitization according to claim 1, wherein
a mass ratio between a total amount of the SIS and the PIB, and the alicyclic saturated hydrocarbon resin ((SIS+PIB):alicyclic saturated hydrocarbon resin) is in a range of 1:1.7 to 1:5.

7. The method for reducing skin sensitization according to claim 1, wherein
a mass ratio between the free asenapine and the alicyclic saturated hydrocarbon resin (free asenapine:alicyclic saturated hydrocarbon resin) is in a range of 1:10 to 1:30.

8. The method for reducing skin sensitization according to claim 1, wherein
a mass ratio between a total amount of the SIS and the PIB, and the liquid paraffin ((SIS+PIB):liquid paraffin) is in a range of 6:1 to 1:1.

9. The method for reducing skin sensitization according to claim 1, wherein
a mass ratio between the free asenapine and the liquid paraffin (free asenapine:liquid paraffin) is in a range of 1:1 to 1:5.
